(19) United States
(12) Patent Application Publication
(10) Pub. No.: US 2015/0328277 A1
(43) Pub. Date: Nov. 19, 2015

(54) ORAL TRANSMUCOSAL DELIVERY OF GLATIRAMER ACETATE

(71) Applicants: Ursula GEISTER, (US); Stephan SCHWEIZER, (US); Martina BUERGER, (US); Ralph STEFAN, (US); Gerarld HUBER, (US); Tanja PRIES, (US)

(72) Inventors: Ursula GEISTER, Blaubeuren (DE); Stephan SCHWEIZER, Ehingen (DE); Martina BUERGER, Ulm (DE); Ralph STEFAN, Ebenweiler (DE); Gerald HUBER, Plüderhausen (DE); Tanja PRIES, Munich (DE)

(73) Assignee: Teva Pharmaceutical Industries, Ltd., Petach-Tikva (IL)

(21) Appl. No.: 14/653,013

(22) PCT Filed: Dec. 20, 2013
(86) PCT No.: PCT/US2013/077041

§ 371 Date: Jun. 17, 2015

Related U.S. Application Data
(60) Provisional application No. 61/745,243, filed on Dec. 21, 2012.

(51) Int. Cl. A61K 38/02; A61K 9/00

(52) U.S. Cl. CPC A61K 38/02; A61K 9/006

(57) ABSTRACT

The present invention provides an oral patch comprising:
a liner; and
a film composition thereon, the film composition comprising glatiramer acetate in an amount from about 10 percent to about 40 percent by weight of the film composition and one or more film forming agents in a total amount from about 40 percent to about 80 percent by weight of the film composition.

This application claims benefit of U.S. Provisional Application No. 61/745,243, filed Dec. 21, 2012, the entire content of which is hereby incorporated by reference herein.

Throughout this application various publications are referenced. The disclosures of these publications in their entireties are hereby incorporated by reference into this application in order to more fully describe the state of the art to which this invention pertains.

### BACKGROUND OF THE INVENTION

### Multiple Sclerosis

Multiple Sclerosis (MS) is a chronic, debilitating disease of the central nervous system (CNS). MS has also been classified as an autoimmune disease. MS disease activity can be monitored by magnetic resonance imaging (MRI) of the brain, accumulation of disability, as well as rate and severity of relapses.

There are five main forms of multiple sclerosis:

### 1) Benign Multiple Sclerosis:

Benign multiple sclerosis is a retrospective diagnosis which is characterized by 1-2 exacerbations with complete recovery, no lasting disability and no disease progression for 10-15 years after the initial onset. Benign multiple sclerosis may, however, progress into other forms of multiple sclerosis.

### 2) Relapsing-Remitting Multiple Sclerosis (RRMS):

Patients suffering from RRMS experience sporadic exacerbations or relapses, as well as periods of remission. Lesions and evidence of axonal loss may or may not be visible on MRI for patients with RRMS.

### 3) Secondary Progressive Multiple Sclerosis (SPMS):

SPMS may evolve from RRMS. Patients afflicted with SPMS have relapses, a diminishing degree of recovery during remissions, less frequent remissions and more pronounced neurological deficits than RRMS patients. Enlarged ventricles, which are markers for atrophy of the corpus callosum, midline center and spinal cord, are visible on MRI of patients with SPMS.

### 4) Primary Progressive Multiple Sclerosis (PPMS);

PPMS is characterized by a steady progression of increasing neurological deficits without distinct attacks or remissions. Cerebral lesions, diffuse spinal cord damage and evidence of axonal loss are evident on the MRI of patients with PPMS.

### 5) Progressive-Relapsing Multiple Sclerosis (PRMS):

PRMS has periods of acute exacerbations while proceeding along a course of increasing neurological deficits without remissions. Lesions are evident on MRI of patients suffering from PRMS (Multiple sclerosis: its diagnosis, symptoms, types and stages, 2003, albany.net/.about.tjc/multiple-sclerosis.html; What are the Types of Multiple Sclerosis?, 2005, <imaginis.com/multiple-sclerosis/types-of-ms.asp? mode=1>).

Chronic progressive multiple sclerosis is a term used to collectively refer to SPMS, PPMS, and PRMS (Types of Multiple Sclerosis (MS), 2005, <themcfox.com/multiple-sclerosis/types-of-ms/types-of-multi-ple-sclerosis.htm>). The relapsing forms of multiple sclerosis are SPMS with superimposed relapses, RRMS and PRMS.

A clinically isolated syndrome (CIS) is a single monosymptomatic attack compatible with MS, such as optic neuritis, brain stem symptoms, and partial myelitis. Patients with CIS that experience a second clinical attack are generally considered to have clinically definite multiple sclerosis (CDMS). Over 80 percent of patients with a CIS and MRI lesions go on to develop MS, while approximately 20 percent have a self-limited process (Frohman et al., The utility of MRI in suspected MS: report of the Therapeutics and Technology Assessment Subcommittee of the American Academy of Neurology, Neurology 61(5):602-11 (2003)).

Multiple sclerosis may present with optic neuritis, blurring of vision, diplopia, involuntary rapid eye movement, blindness, loss of balance, tremors, ataxia, vertigo, clumsiness of a limb, lack of co-ordination, weakness of one or more extremity, altered muscle tone, muscle stiffness, spasms, tingling, paraesthesia, burning sensations, muscle pains, facial pain, trigeminal neuralgia, stabbing sharp pains, burning tingling pain, slowing of speech, slurring of words, changes in rhythm of speech, dysphagia, fatigue, bladder problems (including urgency, frequency, incomplete emptying and incontinence), bowel problems (including constipation and loss of bowel control), impotence, diminished sexual arousal, loss of sensation, sensitivity to heat, loss of short term memory, loss of concentration, or loss of judgment or reasoning.

### Glatiramer Acetate

Glatiramer acetate (GA), a mixture of polypeptides which do not all have the same amino acid sequence, is marketed under the tradename Copaxone®. GA comprises the acetate salts of polypeptides containing L-glutamic acid, L-alanine, L-tyrosine and L-lysine at average molar fractions of 0.141, 0.427, 0.095 and 0.338, respectively. The average molecular weight of Copaxone® is between 5,000 and 9,000 daltons. (“Copaxone”, Physician's Desk Reference, (2005), Medical Economics Co., Inc., (Montvale, N.J.), 3115.) Chemically, glatiramer acetate is designated L-glutamic acid polymer with L-alanine, L-lysine, L-tyrosine, acetate (salt).

Its structural formula is:

### (Glu,Ala,Lys,Tyr).×CH₃COOH

(C₅H₉NO₄.C₃H₇NO₂.C₆H₁₄N₂O₂.C₉H₁₁NO₃).×C₂H₄O₂

### CAS-147245-92-9

Copaxone® (“Copaxone”, Full Prescribing Information, (February, 2009), FDA Marketing Label) (20 mg glatiramer acetate daily injection) is an approved therapy for patients with relapsing remitting multiple sclerosis (RRMS), including patients who have experienced a first clinical episode and have MRI features consistent with multiple sclerosis.

GA has also been disclosed for use in the treatment of other autoimmune diseases (U.S. Patent Publication No. 2002/0055466 A1 (R. Aharoni et al.), inflammatory non-autoimmune diseases (U.S. Patent Publication No. 2005/0014694 A1 (V. Wee Yong et al.); and U.S. Patent Application No. 2002/0077278 A1, published Jun. 20, 2002 (Young et al.)) and other diseases (U.S. Patent Publication Nos. 2003/0004099 A1 and 2002/0037848 A1 (Eisenbach-Schwartz, et al.); U.S. Pat. No. 6,514,938 B1, issued Feb. 4, 2003 (Gad et al.); PCT International Publication No. WO 01/60392, published Aug. 23, 2001 (Gilbert et al.); PCT International Publication No. WO 00/27417, published May 19, 2000 (Aharoni et al.); and PCT International Publication No. WO 01/97846, published Dec. 27, 2001 (Moses et al.).

The 20 mg/day subcutaneous (s.c.) dose has been shown to reduce the total number of enhancing lesions in MS patients as measured by MRI (G. Comi et al., European/Canadian Multicenter, Double-Blind, Randomized, Placebo-Controlled Study of the Effects of Glatiramer Acetere on Magnetic Resonance Imaging-Measured Disease Activity and Burden in Patients with Relapsing Multiple Sclerosis, Ann. Neurol. 49:290-297 (2001)).

Safety data accumulated for GA in clinical trials shows that the drug product is safe and well tolerated. However, reactions including Immediate Post-Injection Reaction (IPIR) consisting of one or more of the following symptoms: vasodilatation, chest pain, dyspnoea, palpitations or tachycardia was reported for 31% of the GA patients vs. 13% on placebo. Additional adverse reactions reported by patients treated with GA 20 mg with at least 2% higher incidence than with placebo were pain, nausea, anxiety, rash, back pain, chills, face edema, local reaction, lymphadenopathy, vomiting, weight increase, tremor, skin disorder, eye disorder, vaginal candidiasis and injection site atrophy.

In all clinical trials, injection-site reactions were seen to be the most frequent adverse reactions and were reported by the majority of patients receiving GA. In controlled studies, the proportion of patients reporting these reactions, at least once, was higher following treatment with GA (70%) than placebo injections (37%). The most commonly reported injection-site reactions, which were more frequently reported in GA vs. placebo-treated patients, were erythema, pain, mass, pruritus, edema, inflammation and hypersensitivity.

In addition to the observed adverse events, administration by injection can be burdensome which can lead to poor patient compliance or suspension of therapy. Accordingly, there exists a need to develop alternative routes of glatiramer acetate delivery in which the glatiramer acetate is effective in treating a symptom of a form of multiple sclerosis.

### Alternatives to Glatiramer Acetate Injection

Glatiramer acetate administration through ingestion or inhalation has been disclosed (U.S. Pat. No. 6,214,791); and compositions for oral, nasal and pulmonary administration also have been disclosed (U.S. Patent Application Publication No. 2001/0055568 A1).

Studies in mice showed that orally administered glatiramer acetate inhibited the induction of experimental autoimmune encephalomyelitis (EAE) in rats and mice and suggested that oral administration of glatiramer acetate may modulate multiple sclerosis as well (Teitelbaum et al., Immunomodulation of experimental autoimmune encephalomyelitis by oral administration of copolymer 1, Immunology 96:3842-3847 (1999)). However, alternative routes of administration have yet to be demonstrated to be effective in the treatment of multiple sclerosis. For example, glatiramer acetate administered orally did not affect relapse rate or other clinical MRI parameters of disease activity in a recent clinical trial (Filippi et al, Effects of oral glatiramer acetate on clinical and MRI-monitored disease activity in patients with relapsing multiple sclerosis: a multicentre, double-blind, randomised, placebo-controlled study, Lancet Neurol. 5(3):213-220 (2006)).

Buccal administration avoids hepatic metabolism and gastrointestinal degradation which can hinder effectiveness of orally administered drugs and provides an attractive alternative to oral administration. However, the buccal mucosa is not an absorptive organ and permeation of the drug to be administered is problematic. Other problems to be overcome include drug stability and formulation palatability.

### Advantages of Mucoadhesive Buccal Drug Delivery System

Drugs administered via oral mucosa offers several ad-vantages
Ease of administration.
Termination of therapy is easy.
Permits localization of drug to the oral cavity for a prolonged period of time.
Can be administered to unconscious patients.
Offers an excellent route, for the systemic delivery of drugs with high first pass metabolism, thereby offering a greater bioavailability.
A significant reduction in dose can be achieved there by reducing dose related side effects.
Drugs which are unstable in the acidic environment are destroyed by enzymatic or alkaline environment of intestine can be administered by this route.
Drugs which show poor bioavailability via the oral route can be administered conveniently.
It offers a passive system of drug absorption and does not require any activation.
The presence of saliva ensures relatively large amount of water for drug dissolution unlike in case of rectal and transdermal routes.
Systemic absorption is rapid.
This route provides an alternative for the administration of various hormones, narcotic analgesic, steroids, enzymes, cardiovascular agents etc.
The buccal mucosa is highly perfused with blood vessels and offers a greater permeability than the skin.

### SUMMARY OF THE INVENTION

The present invention provides an oral patch comprising:
a liner; and
a film composition thereon, the film composition comprising glatiramer acetate in an amount from about 10 percent to about 40 percent by weight of the film composition and one or more film forming agents in a total amount from about 40 percent to about 80 percent by weight of the film composition.

The present invention also provides an oral patch comprising:
a PET liner; and
a film composition thereon, the film composition comprising glatiramer acetate in an amount of about 25 percent by weight of the film composition and film forming agents, wherein the film forming agents comprise:
carbomer (sodium salt) present in the film composition in an amount of about 25.5 percent by weight of the film composition;
polyethylene glycol present in the film composition in an amount of about 2.5 percent by weight of the film composition;
polyvinyl alcohol present in the film composition in an amount of about 10 percent by weight of the film composition; and
microcrystalline cellulose present in the film composition in an amount of about 15 percent by weight of the film composition,
   wherein the film composition further comprises a filler which comprises sorbitol present in the film composition in an amount of about 17 percent by weight of the film composition,
   wherein the film composition further comprises a permeation enhancer which comprises DMSO present in the film composition in an amount of about 2.5 percent by weight of the film composition,
   wherein the film composition further comprises flavorants which comprise:
acesulfam present in the film composition in an amount of about 1.5 percent by weight of the film composition; and
orange flavor present in the film composition in an amount of about 1.5 percent by weight of the film composition,
   wherein the patch is about 10 cm²,
   wherein the patch contains about 25 mg glatiramer acetate.

The present invention also provides an oral patch comprising:
a PET liner; and
a film composition thereon, the film composition comprising glatiramer acetate in an amount of about 23 percent by weight of the film composition and film forming agents, wherein the film forming agents comprise:
carbomer (sodium salt) present in the film composition in an amount of about 27 percent by weight of the film composition;
polyvinyl alcohol present in the film composition in an amount of about 14 percent by weight of the film composition;
polyethylene glycol present in the film composition in an amount of about 3 percent by weight of the film composition; and
rice starch present in the film composition in an amount of about 17 percent by weight of the film composition,
   wherein the film composition further comprises a filler which comprises sorbitol present in the film composition in an amount of about 14 percent by weight of the film composition,
   wherein the film composition further comprises a permeation enhancer which comprises n-Dodecyl nitrogen heterocyclic heptane-2-ketone present in the film composition in an amount of about 1 percent by weight of the film composition,
   wherein the film composition further comprises flavorants which comprise:
acesulfam present in the film composition in an amount of about 0.5 percent by weight of the film composition; and
orange flavor present in the film composition in an amount of about 1 percent by weight of the film composition,
   wherein the patch is about 10 cm²,
   wherein the patch contains about 25 mg glatiramer acetate.

The present invention also provides an oral patch comprising:
a PET liner; and
a film composition thereon, the film composition comprising glatiramer acetate in an amount of about 23 percent by weight of the film composition and film forming agents, wherein the film forming agents comprise:
polyvinyl alcohol present in the film composition in an amount of about 28 percent by weight of the film composition;
polyethylene glycol present in the film composition in an amount of about 3 percent by weight of the film composition;
rice starch present in the film composition in an amount of about 18 percent by weight of the film composition; and
hydroxypropyl methylcellulose present in the film composition in an amount of about 13 percent by weight of the film composition,
   wherein the film composition further comprises a filler which comprises sorbitol present in the film composition in an amount of about 9 percent by weight of the film composition,
   wherein the film composition further comprises flavorants which comprise:
acesulfam present in the film composition in an amount of about 0.5 percent by weight of the film composition;
peppermint oil present in the film composition in an amount of about 1 percent by weight of the film composition; and
glycerol present in the film composition in an amount of about 3.5 percent by weight of the film composition,
   wherein the film composition further comprises a pigment which comprises titanium dioxide present in the film composition in an amount of about 1 percent by weight of the film composition,
   wherein the patch is about 10 cm²,
   wherein the patch contains about 25 mg glatiramer acetate.

The present invention also provides an oral patch comprising:
a PET liner; and
a film composition thereon, the film composition comprising glatiramer acetate in an amount of about 24 percent by weight of the film composition and film forming agents, wherein the film forming agents comprise:
polyvinyl alcohol present in the film composition in an amount of about 10 percent by weight of the film composition;
polyethylene glycol present in the film composition in an amount of about 2.5 percent by weight of the film composition; and
amylopectin present in the film composition in an amount of about 46 percent by weight of the film composition,
   wherein the film composition further comprises a filler which comprises sorbitol present in the film composition in an amount of about 10 percent by weight of the film composition,
   wherein the film composition further comprises permeation enhancers which comprise:
propylene glycol present in the film composition in an amount of about 2.5 percent by weight of the film composition; and
oleic acid present in the film composition in an amount of about 1 percent by weight of the film composition,
   wherein the film composition further comprises flavorants which comprise:
acesulfam present in the film composition in an amount of about 1.5 percent by weight of the film composition; and
spearmint flavor present in the film composition in an amount of about 3 percent by weight of the film composition,
   wherein the patch is about 10 cm²,
   wherein the patch contains about 25 mg glatiramer acetate.

The present invention also provides an oral patch comprising:
a PET liner; and
a film composition thereon, the film composition comprising glatiramer acetate in an amount of about 23 percent by weight and film forming agents, wherein the film forming agents comprise:
polyvinyl alcohol present in the film composition in an amount of about 28 percent by weight of the film composition;
polyethylene glycol present in the film composition in an amount of about 3 percent by weight of the film composition; and
amylopectin present in the film composition in an amount of about 31 percent by weight of the film composition,
   wherein the film composition further comprises a filler which comprises sorbitol present in the film composition in an amount of about 9 percent by weight of the film composition,
   wherein the film composition further comprises flavorants which comprise:
acesulfam present in the film composition in an amount of about 0.5 percent by weight of the film composition;
peppermint oil present in the film composition in an amount of about 1 percent by weight of the film composition; and
glycerol present in the film composition in an amount of about 3.5 percent by weight of the film composition,
   wherein the film composition further comprises a pigment which comprises titanium dioxide present in the film composition in an amount of about 1 percent by weight of the film composition,
   wherein the patch is about 10 cm²,
   wherein the patch contains about 25 mg glatiramer acetate.

### BRIEF DESCRIPTION OF THE DRAWINGS

FIG. 1 shows the average permeation of glatiramer acetate across a buccal membrane. Data series are presented as follows: glatiramer acetate solution without pre-incubated tissue (square markers, solid line) and glatiramer acetate solution with DMSO pre-incubated tissue (square markers, dotted line).

### DETAILED DESCRIPTION OF THE INVENTION

### Terms

As used in this application, except as otherwise expressly provided herein, each of the following terms shall have the meaning set forth below.

As used herein, an “amount” or “dose” of an agent measured in milligrams refers to the milligrams of agent present in a drug product, regardless of the form of the drug product.

Administration of different amounts of glatiramer acetate using oral patches of the present invention can be accomplished by applying one, two, three, four or five oral patches at the same time or consecutively or by applying a portion of an oral patch. For example ½ of an oral patch can be obtained by cutting an oral patch once and ¼ of an oral patch can be obtained by cutting an oral patch twice.

Administration of an amount from about 5 to about 100 mg of glatiramer acetate can be achieved using the oral patches of the present invention. For Example, administration of 5 mg glatiramer acetate can be accomplished by applying ¼ of an oral patch containing 20 mg glatiramer acetate and administration of 10 mg glatiramer acetate can be accomplished by applying ½ of an oral patch containing 20 mg glatiramer acetate. Likewise, administration of 20, 40, 60, 80 or 100 mg glatiramer acetate can be accomplished, for example, by applying 1, 2, 3, 4 or 5 oral patches containing 20 mg glatiramer acetate, respectively. Similarly, administration of 100 mg glatiramer acetate can be accomplished, for example, by applying a single oral patch containing 100 mg glatiramer acetate, or by applying 2 oral patches containing 50 mg glatiramer acetate, etc.

As used herein, the term “composition”, as in pharmaceutical composition, is intended to encompass a product comprising the active ingredient(s) and the inert ingredient(s) that make up the carrier, as well as any product which results, directly or indirectly from combination, complexation, or aggregation of any two or more of the ingredients, or from dissociation of one or more of the ingredients, or from other types of reactions or interactions of one or more of the ingredients.

As used herein, “film forming agents” are agents which form a matrix which allows for controlled release of an active ingredient. Film forming agents include, but are not limited to, Carbomer (sodium salt), polyethylene glycol, polyvinyl alcohol, microcrystalline cellulose, starch, hydroxypropyl methylcellulose, amylopectin, ethylcellulose, gelatine, hydroxypropylcellulose, hydroxyethylcellulose, methylcellulose, carboxymethylcellulose, gummi arabicum, xanthan gum and carrageen.

As used herein, “permeation enhancers” are agents which increase bioavailability of the active ingredient. Permeation enhancers include, but are not limited to, DMSO, n-Dodecyl nitrogen heterocyclic heptane-2-ketone, propylene glycol, isopropylmyristat, d,l-alpha-toccopherol and oleic acid.

As used herein, “flavourant” include sweeteners including but not limited to acesulfam, saccharin-sodium, aspartame, and stevia. Other suitable flavourants can include, for example, flavors, which are known to those of skill in the art, such as, for example, natural flavors, artificial flavors, and combinations thereof. Flavourants are compatible with the ingredients of the pharmaceutical composition, i.e., they do not substantially reduce the chemical stability, the physical stability, or the biological activity of the pharmaceutical composition. Flavoring agents may be chosen, e.g., from synthetic flavor oils and flavoring aromatics and/or oils, oleoresins, extracts derived from plants, leaves, flowers, fruits, and the like, and combinations thereof. Non-limiting examples of flavor oils include spearmint oil, cinnamon oil, oil of wintergreen (methyl salicylate), peppermint oil, clove oil, bay oil, anise oil, eucalyptus oil, thyme oil, cedar leaf oil, oil of nutmeg, allspice, oil of sage, mace, oil of bitter almonds, and cassia oil. Suitable flavoring agents also include, for example, artificial, natural and synthetic flower derived or fruit flavors such as vanilla, ethyl vanillin, citrus oils (e.g., lemon, orange, tangerine, lime, and grapefruit), and fruit essences (e.g., natural and/or artificial flavor of apple, pear, peach, orange, grape, strawberry, raspberry, cherry, plum, pineapple, and apricot), and the like, and combinations thereof. The flavourants may be used in liquid or solid form and, as indicated above, may be used individually or in admixture. Other flavourants can include, for example, certain aldehydes and esters, e.g., cinnamyl acetate, cinnamaldehyde, citral diethylacetal, dihydrocarvyl acetate, eugenyl formate, p-methylamisol, and the like, and combinations thereof. They can be liquids or spray-dried, co-processed powders.

Pharmaceutical compositions of the present invention can optionally comprise one or more colorants, flavors, and/or fragrances to enhance the visual appeal, taste, and/or scent of the composition. Suitable colorants, flavors, or fragrances are compatible with the ingredients of the pharmaceutical composition, i.e., they do not substantially reduce the solubility, the chemical stability, the physical stability or the biological activity of the pharmaceutical composition. In one embodiment, the pharmaceutical composition comprises a colorant, a flavor, and/or a fragrance. For example, the pharmaceutical composition comprises less than about 1 wt % (e.g., less than about 0.75 wt % or less than about 0.5 wt %) of each optionally ingredient, i.e., colorant, flavor and/or fragrance, by weight of the composition. In another example, the pharmaceutical composition comprises less than about 1 wt % (e.g., less than about 0.75 wt % or less than about 0.5 wt %) of a colorant. In still another example, the pharmaceutical composition comprises less than about 1 wt % (e.g., less than about 0.75 wt % or less than about 0.5 wt %) of a blue colorant (e.g., FD&C Blue #1 and/or FD&C Blue #2 Aluminum Lake, commercially available from Colorcon, Inc. of West Point, Pa.)

As used herein, colorants can include, but are not limited to, Annatto extract, Dehydrated beets (beet powder), Canthaxanthin, Caramel, β-Apo-8′-carotenal, β-Carotene, Cochineal extract, Carmine, Sodium copper chlorophyllin, Toasted partially defatted cooked cottonseed flour, Ferrous gluconate, Ferrous lactate, Grape color extract, Grape skin extract (enocianina), Synthetic iron oxide, Fruit juice, Vegetable juice, Carrot oil, Paprika, Paprika oleoresin, Mica-based pearlescent pigments, Riboflavin, Saffron, Titanium dioxide, Tomato lycopene extract; tomato lycopene concentrate, Turmeric, Turmeric oleoresin, FD&C Blue No. 1, FD&C Blue No. 2, FD&C Green No. 3, Orange B, Citrus Red No. 2, FD&C Red No. 3, FD&C Red No. 40, FD&C Yellow No. 5, FD&C Yellow No. 6, Alumina (dried aluminum hydroxide), Calcium carbonate, Canthaxanthin, Potassium sodium copper chlorophyllin (chlorophyllin-copper complex), Dihydroxyacetone, Bismuth oxychloride, Synthetic iron oxide, Ferric ammonium ferrocyanide, Ferric ferrocyanide, Chromium hydroxide green, Chromium oxide greens, Guanine, Pyrophyllite, Mica, Talc, Aluminum powder, Bronze powder, Copper powder, Zinc oxide, D&C Blue No. 4, D&C Green No. 6, D&C Green No. 8, D&C Orange No. 4, D&C Orange No. 5, D&C Orange No. 10, D&C Orange No. 11, FD&C Red No. 4, D&C Red No. 6, D&C Red No. 7, D&C Red No. 17, D&C Red No. 21, D&C Red No. 22, D&C Red No. 27, D&C Red No. 28, D&C Red No. 30, D&C Red No. 31, D&C Red No. 33, D&C Red No. 34, D&C Red No. 36, D&C Red No. 39, D&C Violet No. 2, D&C Yellow No. 7, Ext. D&C Yellow No. 7, D&C Yellow No. 8, D&C Yellow No. 10 and D&C Yellow No. 11.

As used herein, a “perfusion enhancer” is an agent which increases blood flow to the capillary beds. Perfusion enhancers can include, but are not limited to, capsaicin and apitoxin and DMSO.

Acetic acid, as used herein, is an added in order to provoke a faster onset of an immune reaction.

Relapsing Form of Multiple Sclerosis:

The term relapsing MS includes:
1) patients with RRMS;
2) patients with SPMS and superimposed relapses; and
3) patients with CIS who show lesion dissemination on subsequent MRI scans according to McDonald's criteria.

As used herein, relapsing forms of multiple sclerosis include: Relapsing-remitting multiple sclerosis (RRMS), characterized by unpredictable acute episodes of neurological dysfunction (relapses), followed by variable recovery and periods of clinical stability;

Secondary Progressive MS (SPMS), wherein patients having RRMS develop sustained deterioration with or without relapses superimposed; and

Primary progressive-relapsing multiple sclerosis (PPRMS) or progressive-relapsing multiple sclerosis (PRMS), an uncommon form wherein patients developing a progressive deterioration from the beginning can also develop relapses later on.

### EMBODIMENTS OF THE INVENTION

The present invention provides an oral patch comprising:
a liner; and
a film composition thereon, the film composition comprising glatiramer acetate in an amount from about 10 percent to about 40 percent by weight of the film composition and one or more film forming agents in a total amount from about 40 percent to about 80 percent by weight of the film composition.

In one or more embodiments of the present invention, the glatiramer acetate is present in the film composition in an amount from about 20 percent to about 30 percent by weight of the film composition.

In one or more embodiments of the present invention, the glatiramer acetate is present in the film composition in an amount from about 22 percent to about 27 percent by weight of the film composition.

In one or more embodiments of the present invention, the glatiramer acetate is present in the film composition in an amount of about 23-25 percent by weight of the film composition.

In one or more embodiments of the present invention, the glatiramer acetate is present in the film composition in an amount of about 23 percent by weight of the film composition.

In one or more embodiments of the present invention, the glatiramer acetate is present in the film composition in an amount of about 24 percent by weight of the film composition.

In one or more embodiments of the present invention, the glatiramer acetate is present in the film composition in an amount of about 25 percent by weight of the film composition.

In one or more embodiments of the present invention, the film forming agents are present in the film composition in a total amount from about 50 percent to about 70 percent by weight of the film composition.

In one or more embodiments of the present invention, the film composition further comprises a filler, wherein the filler is present in the film composition in an amount up to about 30 percent by weight of the film composition.

In one or more embodiments of the present invention, the filler is present in the film composition in an amount from about 5 percent to about 25 percent by weight of the film composition.

In one or more embodiments of the present invention, the filler is present in the film composition in an amount from about 9 percent to about 17 percent by weight of the film composition.

In one or more embodiments of the present invention, the filler is present in the film composition in an amount of about 9 percent by weight of the film composition.

In one or more embodiments of the present invention, the filler is present in the film composition in an amount of about 10 percent by weight of the film composition.

In one or more embodiments of the present invention, the filler is present in the film composition in an amount of about 14 percent by weight of the film composition.

In one or more embodiments of the present invention, the filler is present in the film composition in an amount of about 17 percent by weight of the film composition.

In one or more embodiments of the present invention, the film composition further comprises one or more permeation enhancers, wherein the permeation enhancers are present in the film composition in a total amount up to about 10 percent by weight of the film composition.

In one or more embodiments of the present invention, the permeation enhancers are present in the film composition in a total amount from about 0.1 percent to about 7 percent by weight of the film composition.

In one or more embodiments of the present invention, the permeation enhancers are present in the film composition in a total amount from about 0.5 percent to about 5 percent by weight of the film composition.

In one or more embodiments of the present invention, the film composition further comprises one or more flavorant, wherein the flavorants are present in the film composition in a total amount up to about 10 percent by weight of the film composition.

In one or more embodiments of the present invention, the film composition further comprises a pigment, wherein the pigment are present in the film composition in an amount up to about 5 percent by weight of the film composition.

In one or more embodiments of the present invention, the pigment is present in the film composition in an amount of about 1 percent by weight of the film composition.

In one or more embodiments of the present invention, the one or more film forming agents are selected from the group consisting of Carbomer (sodium salt), polyethylene glycol, polyvinyl alcohol, microcrystalline cellulose, starch, hydroxypropyl methylcellulose and amylopectin.

In one or more embodiments of the present invention, the filler is selected from the group consisting of sorbitol, lactose, saccharose, sucrose, dextrose, isomalt calcium phosphate, calcium carbonate, calcium silicate, magnesium carbonate, magnesium oxide, glucopyranosyl mannitol and calcium sulfate.

In one or more embodiments of the present invention, the one or more permeation enhancers are selected from the group consisting of DMSO, n-Dodecyl nitrogen heterocyclic heptane-2-ketone, propylene glycol, oleic acid, isopropylmyristat and d,l-alpha-toccopherol.

In one or more embodiments of the present invention, the one or more flavorants are selected from the group consisting of acesulfam, saccharin-sodium, aspartame, stevia, spearmint oil, cinnamon oil, oil of wintergreen (methyl salicylate), peppermint oil, clove oil, bay oil, anise oil, eucalyptus oil, thyme oil, cedar leaf oil, oil of nutmeg, allspice, oil of sage, mace, oil of bitter almonds, cassia oil, vanilla, ethyl vanillin, citrus oils, lemon oil, orange oil, tangerine oil, lime oil, grapefruit oil, apple flavor, pear flavor, peach flavor, orange flavor, grape flavor, strawberry flavor, raspberry flavor, cherry flavor, plum flavor, pineapple flavor, apricot flavor, cinnamyl acetate, cinnamaldehyde, citral diethylacetal, dihydrocarvyl acetate, eugenyl formate and p-methylamisol.

In one or more embodiments of the present invention, the pigment is selected from the group consisting of titanium dioxide, talc and ferric oxide.

In one or more embodiments of the present invention, the one or more film forming agents comprises carbomer (sodium salt), wherein the carbomer (sodium salt) is present in the film composition in an amount from about from about 20 percent to about 35 percent by weight of the film composition.

In one or more embodiments of the present invention, the carbomer (sodium salt) is present in the film composition in an amount from about from about 25 percent to about 30 percent by weight of the film composition.

In one or more embodiments of the present invention, the carbomer (sodium salt) is present in the film composition in an amount of about 25.5 percent by weight of the film composition.

In one or more embodiments of the present invention, the carbomer (sodium salt) is present in the film composition in an amount of about 27 percent by weight of the film composition.

In one or more embodiments of the present invention, the one or more film forming agents comprises polyethylene glycol, wherein the polyethylene glycol is present in the film composition in an amount from about from about 0.5 percent to about 5 percent by weight of the film composition.

In one or more embodiments of the present invention, the polyethylene glycol is present in the film composition in an amount of about 2 percent by weigh of the film composition.

In one or more embodiments of the present invention, the polyethylene glycol is present in the film composition in an amount of about 2.5 percent by weigh of the film composition.

In one or more embodiments of the present invention, the polyethylene glycol is present in the film composition in an amount of about 3 percent by weigh of the film composition.

In one or more embodiments of the present invention, the one or more film forming agents comprises polyvinyl alcohol, wherein the polyvinyl alcohol is present in the film composition in an amount from about from about 5 percent to about 40 percent by weight of the film composition.

In one or more embodiments of the present invention, the polyvinyl alcohol is present in the film composition in an amount from about from about 10 percent to about 30 percent by weight of the film composition.

In one or more embodiments of the present invention, the polyvinyl alcohol is present in the film composition in an amount from about from about 15 percent to about 28 percent by weight of the film composition.

In one or more embodiments of the present invention, the polyvinyl alcohol is present in the film composition in an amount of about 10 percent by weight of the film composition.

In one or more embodiments of the present invention, the polyvinyl alcohol is present in the film composition in an amount of about 14 percent by weight of the film composition.

In one or more embodiments of the present invention, the polyvinyl alcohol is present in the film composition in an amount of about 28 percent by weight of the film composition.

In one or more embodiments of the present invention, the one or more film forming agents comprises microcrystalline cellulose, wherein microcrystalline cellulose is present in the film composition in an amount from about from about 5 percent to about 25 percent by weight of the film composition.

In one or more embodiments of the present invention, the microcrystalline cellulose is present in the film composition in an amount of about 15 percent by weight of the film composition.

In one or more embodiments of the present invention, the one or more film forming agents comprises polyethylene glycol, wherein the polyethylene glycol is present in the film composition in an amount from about from about 0.5 percent to about 5 percent by weight of the film composition.

In one or more embodiments of the present invention, the polyethylene glycol is present in the film composition in an amount from about from about 2 percent to about 3 percent by weight of the film composition.

In one or more embodiments of the present invention, ein the one or more film forming agents comprises starch, wherein the starch is present in the film composition in an amount from about from about 10 percent to about 20 percent by weight of the film composition.

In one or more embodiments of the present invention, the starch is present in the film composition in an amount from about from about 17 percent to about 18 percent by weight of the film composition.

In one or more embodiments of the present invention, the starch is present in the film composition in an amount of about 17 percent by weight of the film composition.

In one or more embodiments of the present invention, the starch is present in the film composition in an amount of about 18 percent by weight of the film composition.

In one or more embodiments of the present invention, the one or more film forming agents comprises hydroxypropyl methylcellulose, wherein the hydroxypropyl methylcellulose is present in the film composition in an amount from about from about 10 percent to about 15 percent by weight of the film composition.

In one or more embodiments of the present invention, the hydroxypropyl methylcellulose is present in the film composition in an amount of about 13 percent by weight of the film composition.

In one or more embodiments of the present invention, the one or more film forming agents comprises amylopectin, wherein the amylopectin is present in the film composition in an amount from about from about 25 percent to about 55 percent by weight of the film composition.

In one or more embodiments of the present invention, the amylopectin is present in the film composition in an amount from about from about 31 percent to about 47 percent by weight of the film composition.

In one or more embodiments of the present invention, the amylopectin is present in the film composition in an amount of about 31 percent by weight of the film composition.

In one or more embodiments of the present invention, the amylopectin is present in the film composition in an amount of about 47 percent by weight of the film composition.

In one or more embodiments of the present invention, the one or more permeation enhancers comprises DMSO, wherein the DMSO is present in the film composition in an amount from about from about 0.5 percent to about 5 percent by weight of the film composition.

In one or more embodiments of the present invention, the DMSO is present in the film composition in an amount of about 2.5 percent by weight of the film composition.

In one or more embodiments of the present invention, the one or more permeation enhancers comprises n-Dodecyl nitrogen heterocyclic heptane-2-ketone, wherein the n-Dodecyl nitrogen heterocyclic heptane-2-ketone is present in the film composition in an amount from about from about 0.5 percent to about 5 percent by weight of the film composition.

In one or more embodiments of the present invention, the n-Dodecyl nitrogen heterocyclic heptane-2-ketone is present in the film composition in an amount of about 1 percent by weight of the film composition.

In one or more embodiments of the present invention, the one or more permeation enhancers comprises propylene glycol, wherein the propylene glycol is present in the film composition in an amount from about from about 0.5 percent to about 5 percent by weight of the film composition.

In one or more embodiments of the present invention, the propylene glycol is present in the film composition in an amount of about 2.5 percent by weight of the film composition.

In one or more embodiments of the present invention, the one or more permeation enhancers comprises oleic acid, wherein the oleic acid is present in the film composition in an amount from about from about 0.5 percent to about 5 percent by weight of the film composition.

In one or more embodiments of the present invention, the oleic acid is present in the film composition in an amount of about 1 percent by weight of the film composition.

In one or more embodiments of the present invention, the one or more flavorants comprises acesulfam, wherein the acesulfam is present in the film composition in an amount from about from about 0.25 percent to about 2.5 percent by weight of the film composition.

In one or more embodiments of the present invention, the acesulfam is present in the film composition in an amount from about from about 0.5 percent to about 1.5 percent by weight of the film composition.

In one or more embodiments of the present invention, the acesulfam is present in the film composition in an amount of about 0.5 percent by weight of the film composition.

In one or more embodiments of the present invention, the acesulfam is present in the film composition in an amount of about 1.5 percent by weight of the film composition.

In one or more embodiments of the present invention, the one or more flavorants comprises orange flavor, wherein the orange flavor is present in the film composition in an amount from about from about 0.25 percent to about 2.5 percent by weight of the film composition.

In one or more embodiments of the present invention, the orange flavor is present in the film composition in an amount of about 1 percent by weight of the film composition.

In one or more embodiments of the present invention, the one or more flavorants comprises peppermint oil, wherein the peppermint oil is present in the film composition in an amount from about from about 0.25 percent to about 2.5 percent by weight of the film composition.

In one or more embodiments of the present invention, the peppermint oil is present in the film composition in an amount of about 1 percent by weight of the film composition.

In one or more embodiments of the present invention, the one or more flavorants comprises glycerol, wherein the glycerol is present in the film composition in an amount from about from about 0.5 percent to about 10 percent by weight of the film composition.

In one or more embodiments of the present invention, the glycerol is present in the film composition in an amount of about 4 percent by weight of the film composition.

In one or more embodiments of the present invention, the one or more flavorants comprises spearmint oil, wherein the spearmint oil is present in the film composition in an amount from about from about 0.5 percent to about 5 percent by weight of the film composition.

In one or more embodiments of the present invention, the spearmint oil is present in the film composition in an amount of about 3 percent by weight of the film composition.

In one or more embodiments of the present invention, the patch is about 5 to about 15 cm².

In one or more embodiments of the present invention, the patch is about 10 cm².

In one or more embodiments of the present invention, the patch contains about 20 mg to about 100 mg glatiramer acetate.

In one or more embodiments of the present invention, the patch contains about 25 mg glatiramer acetate.

In one or more embodiments of the present invention, the liner is a polyethylene terephthalate (PET) liner.

The present invention also provides an oral patch comprising:
a PET liner; and
a film composition thereon, the film composition comprising glatiramer acetate in an amount of about 25 percent by weight of the film composition and film forming agents, wherein the film forming agents comprise:
carbomer (sodium salt) present in the film composition in an amount of about 25.5 percent by weight of the film composition;
polyethylene glycol present in the film composition in an amount of about 2.5 percent by weight of the film composition;
polyvinyl alcohol present in the film composition in an amount of about 10 percent by weight of the film composition; and
microcrystalline cellulose present in the film composition in an amount of about 15 percent by weight of the film composition,
   wherein the film composition further comprises a filler which comprises sorbitol present in the film composition in an amount of about 17 percent by weight of the film composition,
   wherein the film composition further comprises a permeation enhancer which comprises DMSO present in the film composition in an amount of about 2.5 percent by weight of the film composition,
   wherein the film composition further comprises flavorants which comprise:
acesulfam present in the film composition in an amount of about 1.5 percent by weight of the film composition; and
orange flavor present in the film composition in an amount of about 1.5 percent by weight of the film composition,
   wherein the patch is about 10 cm²,
   wherein the patch contains about 25 mg glatiramer acetate.

The present invention also provides an oral patch comprising:
a PET liner; and
a film composition thereon, the film composition comprising glatiramer acetate in an amount of about 23 percent by weight of the film composition and film forming agents, wherein the film forming agents comprise:
carbomer (sodium salt) present in the film composition in an amount of about 27 percent by weight of the film composition;
polyvinyl alcohol present in the film composition in an amount of about 14 percent by weight of the film composition;
polyethylene glycol present in the film composition in an amount of about 3 percent by weight of the film composition; and
rice starch present in the film composition in an amount of about 17 percent by weight of the film composition,
   wherein the film composition further comprises a filler which comprises sorbitol present in the film composition in an amount of about 14 percent by weight of the film composition,
   wherein the film composition further comprises a permeation enhancer which comprises n-Dodecyl nitrogen heterocyclic heptane-2-ketone present in the film composition in an amount of about 1 percent by weight of the film composition,
   wherein the film composition further comprises flavorants which comprise:
acesulfam present in the film composition in an amount of about 0.5 percent by weight of the film composition; and
orange flavor present in the film composition in an amount of about 1 percent by weight of the film composition,
   wherein the patch is about 10 cm²,
   wherein the patch contains about 25 mg glatiramer acetate.

The present invention also provides an oral patch comprising:
a PET liner; and
a film composition thereon, the film composition comprising glatiramer acetate in an amount of about 23 percent by weight of the film composition and film forming agents, wherein the film forming agents comprise:
polyvinyl alcohol present in the film composition in an amount of about 28 percent by weight of the film composition;
polyethylene glycol present in the film composition in an amount of about 3 percent by weight of the film composition;
rice starch present in the film composition in an amount of about 18 percent by weight of the film composition; and
hydroxypropyl methylcellulose present in the film composition in an amount of about 13 percent by weight of the film composition,
   wherein the film composition further comprises a filler which comprises sorbitol present in the film composition in an amount of about 9 percent by weight of the film composition,
   wherein the film composition further comprises flavorants which comprise:
acesulfam present in the film composition in an amount of about 0.5 percent by weight of the film composition;
peppermint oil present in the film composition in an amount of about 1 percent by weight of the film composition; and
glycerol present in the film composition in an amount of about 3.5 percent by weight of the film composition,
   wherein the film composition further comprises a pigment which comprises titanium dioxide present in the film composition in an amount of about 1 percent by weight of the film composition,
   wherein the patch is about 10 cm²,
   wherein the patch contains about 25 mg glatiramer acetate.

The present invention also provides an oral patch comprising:
a PET liner; and
a film composition thereon, the film composition comprising glatiramer acetate in an amount of about 24 percent by weight of the film composition and film forming agents, wherein the film forming agents comprise:
polyvinyl alcohol present in the film composition in an amount of about 10 percent by weight of the film composition;
polyethylene glycol present in the film composition in an amount of about 2.5 percent by weight of the film composition; and
amylopectin present in the film composition in an amount of about 46 percent by weight of the film composition,
   wherein the film composition further comprises a filler which comprises sorbitol present in the film composition in an amount of about 10 percent by weight of the film composition,
   wherein the film composition further comprises permeation enhancers which comprise:
propylene glycol present in the film composition in an amount of about 2.5 percent by weight of the film composition; and
oleic acid present in the film composition in an amount of about 1 percent by weight of the film composition,
   wherein the film composition further comprises flavorants which comprise:
acesulfam present in the film composition in an amount of about 1.5 percent by weight of the film composition; and
spearmint flavor present in the film composition in an amount of about 3 percent by weight of the film composition,
   wherein the patch is about 10 cm²,
   wherein the patch contains about 25 mg glatiramer acetate.

The present invention also provides an oral patch comprising:
a PET liner; and
a film composition thereon, the film composition comprising glatiramer acetate in an amount of about 23 percent by weight and film forming agents, wherein the film forming agents comprise:
polyvinyl alcohol present in the film composition in an amount of about 28 percent by weight of the film composition;
polyethylene glycol present in the film composition in an amount of about 3 percent by weight of the film composition; and
amylopectin present in the film composition in an amount of about 31 percent by weight of the film composition,
   wherein the film composition further comprises a filler which comprises sorbitol present in the film composition in an amount of about 9 percent by weight of the film composition,
   wherein the film composition further comprises flavorants which comprise:
acesulfam present in the film composition in an amount of about 0.5 percent by weight of the film composition;
peppermint oil present in the film composition in an amount of about 1 percent by weight of the film composition; and
glycerol present in the film composition in an amount of about 3.5 percent by weight of the film composition,
   wherein the film composition further comprises a pigment which comprises titanium dioxide present in the film composition in an amount of about 1 percent by weight of the film composition,
   wherein the patch is about 10 cm²,
   wherein the patch contains about 25 mg glatiramer acetate.

As used herein, “about” with regard to a stated number encompasses a range of +10 percent to −10 percent of the stated value. By way of example, about 100 mg therefore includes the range 90-110 mg and therefore also includes 90, 91, 92, 93, 94, 95 96, 97, 98, 99, 100, 101, 102, 103, 104, 105, 106, 107, 108, 109 and 110 mg. Accordingly, about 100 mg includes, in an embodiment, 100 mg.

It is understood that where a parameter range is provided, all integers within that range, tenths thereof, and hundredths thereof, are also provided by the invention. For example, “0.2-5 mg” is a disclosure of 0.2 mg, 0.21 mg, 0.22 mg, 0.23 mg etc. up to 0.3 mg, 0.31 mg, 0.32 mg, 0.33 mg etc. up to 0.4 mg, 0.5 mg, 0.6 mg etc. up to 5.0 mg.

All combinations of the various elements described herein are within the scope of the invention.

This invention is illustrated in the Experimental Details section which follows. This section is set forth to aid in an understanding of the invention but is not intended to, and should not be construed to; limit in any way the invention as set forth in the claims which follow thereafter.

### EXAMPLES

### Example 1

Oral patches are prepared with the film composition set forth in Table 1.

TABLE 1

| Ingredient | mg/10 cm² |
|---|---|
| Glatiramer acetate | 25.00 |
| Carbomer, sodium salt | 25.50 |
| Macrogol 1500 | 2.50 |
| Polyvinyl alcohol | 10.00 |
| Sorbitol | 17.00 |
| DMSO | 2.50 |
| Microcrystalline cellulose | 15.00 |
| Acesulfam | 1.50 |
| Orange flavor, liquid | 1.00 |
| Water (solvent, removed during mfg. process) | — |
| Ethanol (solvent, removed during mfg. process) | — |
| Total | 100.00 |

Polyvinyl alcohol is dissolved in heated water, after cooling down Polyethelyne glycol (PEG, Macrogol), Sorbitol, flavor and Acesulfam are added and stirred until dissolved. Glatiramer acetate and DMSO are dissolved in Ethanol and added to the water solution. Microcrystalline cellulose is added and Carbomer is added while continuously stirring the solution. The stirring was continued until the coating process. After coating with an adequate wet coating thickness on a Polyethylene terephthalate (PET) Liner the film was dried in a cabinet dryer. After drying the films were cut out to single dose units and put in an adequate secondary packaging material.

### Example 2

Oral patches are prepared with the film composition set forth in Table 2.

TABLE 2

| Ingredient | mg/10 cm² |
|---|---|
| Glatiramer acetate | 25.00 |
| Polyvinyl alcohol | 15.00 |
| Carbomer, sodium salt | 30.00 |
| Sorbitol | 15.00 |
| Azone ® | 1.00 |
| Acesulfam | 0.50 |
| Orange flavor, liquid | 1.00 |
| Rice starch | 19.00 |
| Macrogol 1000 | 3.00 |
| Water (solvent, removed during mfg. process) | — |
| Ethanol (solvent, removed during mfg. process) | — |
| Total | 109.50 |

Polyvinyl alcohol is dissolved in heated water, after cooling down PEG, Sorbitol, flavor and Acesulfam is added and stirred until dissolved. Glatiramer acetate and n-Dodecyl nitrogen heterocyclic heptane-2-ketone (Azone) are dissolved in Ethanol and added to the water solution. Rice starch is added and Carbomer is added while continuously stirring the solution. The stirring is continued until the coating process. After coating with an adequate wet coating thickness on a PET Liner the film is dried in a cabinet dryer. After drying the films are cut out to single dose units and put in an adequate secondary packaging material.

### Example 3

Oral patches are prepared with the film composition set forth in Table 3.

TABLE 3

| Ingredient | mg/10 cm² |
|---|---|
| Glatiramer acetate | 25.00 |
| Polyvinyl alcohol | 30.00 |
| Hypromellose, 3 mPas | 14.00 |
| Sorbitol | 10.00 |
| Rice starch | 20.00 |
| Acesulfam | 0.50 |
| Peppermint oil | 1.00 |
| Macrogol 1000 | 3.00 |
| Glycerol | 4.00 |
| Titanium dioxide | 1.00 |
| Water (solvent, removed during mfg. process) | — |
| Ethanol (solvent, removed during mfg. process) | — |
| Total | 108.50 |

Polyvinyl alcohol is dissolved in heated water, after cooling down PEG, Sorbitol, Glycerol, peppermint oil and Acesulfam is added and stirred until dissolved. Titanium is added and stirred continuously. Glatiramer acetate is dissolved in Ethanol and added to the water solution. Rice starch is added and Hydroxypropyl methylcellulose (HPMC, Hypromellose) is added while continuously stirring the solution. The stirring is continued until the coating process. After coating with an adequate wet coating thickness on a PET Liner the film is dried in a cabinet dryer. After drying the films are cut out to single dose units and put in an adequate secondary packaging material.

### Example 4

Oral patches are prepared with the film composition set forth in Table 4.

TABLE 4

| Ingredient | mg/10 cm² |
|---|---|
| Glatiramer acetate | 25.00 |
| Amylopectin (Proloc ® 15) | 48.50 |
| Sorbitol | 10.00 |
| Oleic acid | 1.00 |
| Propylene glycol | 2.50 |
| Polyvinyl alcohol | 11.00 |
| Macrogol 1000 | 2.50 |
| Acesulfam | 1.50 |
| Spearmint flavor, liquid | 3.00 |
| Water (solvent, removed during mfg. process) | — |
| Ethanol (solvent, removed during mfg. process) | — |
| Total | 105.00 |

Polyvinyl alcohol is dissolved in heated water, after cooling down Sorbitol, PEG, Oleic acid, Acesulfam, PG and flavor is added and stirred until dissolved. Titanium is added and stirred continuously. Glatiramer acetate is dissolved in Ethanol and added to the water solution. Amylopectin is added while continuously stirring the solution. The stirring is continued until the coating process. After coating with an adequate wet coating thickness on a PET Liner the film is dried in a cabinet dryer. After drying the films are cut out to single dose units and put in an adequate secondary packaging material.

### Example 5

Oral patches are prepared with the film composition set forth in Table 5.

TABLE 5

| Ingredient | mg/10 cm² |
|---|---|
| Glatiramer acetate | 25.00 |
| Polyvinyl alcohol | 30.00 |
| Amylopectin (Proloc ® 15) | 34.00 |
| Sorbitol | 10.00 |
| Acesulfam | 0.50 |
| Peppermint oil | 1.00 |
| Macrogol 1000 | 3.00 |
| Glycerol | 4.00 |
| Titanium dioxide | 1.00 |
| Water (solvent, removed during mfg. process) | — |
| Ethanol (solvent, removed during mfg. process) | — |
| Total | 108.50 |

Polyvinyl alcohol is dissolved in heated water, after cooling down PEG, Sorbitol, Glycerol, peppermint oil and Acesulfam are added and stirred until dissolved. Titanium is added and stirred continuously. Glatiramer acetate is dissolved in Ethanol and added to the water solution. Amylopectin is added while continuously stirring the solution. The stirring is continued until the coating process. After coating with an adequate wet coating thickness on a PET Liner the film is dried in a cabinet dryer. After drying the films are cut out to single dose units and put in an adequate secondary packaging material.

### Example 6

Oral patches are prepared according to Examples 1-5, above. A batch of patches is stored at room temperature (about 25° C.) and under refrigeration (about 4° C.). Samples from each batch are periodically examined for stability of the glatiramer acetate. The results demonstrate that glatiramer acetate stability in the oral patches of the present invention is acceptable.

### Example 7

Oral patches are prepared according to Examples 1-5, above. Oral patches are placed on one side of a sample of porcine buccal tissue in a Franz cell. Media from the acceptor compartment on the other side of the buccal tissue is sampled and permeability of glatiramer acetate is assessed. The results demonstrate permeation of glatiramer acetate across a sample of buccal tissue.

### Example 8

Oral patches are prepared according to Examples 1-5, above. Oral patches are placed in apparatus 1 or apparatus 2 according to USP and dissolution of the drug is measured. After 15 minutes 85% of the drug is released. The results demonstrate that release of glatiramer acetate stability from the oral patches of the present invention is acceptable.

### Second Series of Experiments

### Example 9

### Transport and Preparation of the Skin

Porcine buccal tissue was obtained from a slaughterhouse. Immediately after slaughter of the pig, pieces bearing the buccal tissue were dissected from the cheek and stored in PBS pH 7.4 and cooled on ice. The buccal tissue was isolated from the inner cheek with a scalpel and used fresh. Subsequently, the suitability of the tissue biopsy was assessed. The exclusion criteria were tissue damage or scarring.

Freshly prepared buccal tissue was cut into stripes. Tissue sections with a thickness of approx. 700-800 μm were then prepared. The dermatome was applied to the buccal tissue surface and the tissue was cut with 24 mm punch.

### Permeation Study

The cylindrical Franz cell is a diffusion chamber comprising an upper and a lower part between which the porcine buccal tissue was clamped. The two halves of the cell were held together by means of a ball and socket clamp. The lower (acceptor) chamber has a volume of approx. 12 ml, while the volume of the upper (donor) chamber is variable. The tissue specimens are punched out immediately prior to insertion in the Franz cells. The tissue is always inserted with the connective tissue (lamina propia and submucosa) facing downwards so that the mucosal epithelium layer is uppermost.

The medium temperature was adjusted to 37° C. and continuously stirred at a rate of 400 rpm. The diffusion area of the porcine buccal tissue in the Franz cell was approx. 1.77 cm².

Experiments utilizing the GA solution were performed in triplicate. For each replicate, 300 μl of the formulation was applied per 1.77 cm² porcine buccal tissue at the start of the experiment. For experiments with permeation enhancer 100 μl DMSO was applied to the buccal tissue 30 minutes before the glatiramer acetate solution was applied because glatiramer acetate is not soluble in a mixture of DMSO/PBS 50:50 v/v %.

Permeation through the porcine buccal tissue into the acceptor medium was monitored over a period of 4 hours. The acceptor medium was sampled at 6 different points of time (30, 60, 90, 120, 180 and 240 min).

### Determination of Glatiramer Acetate

Table 6 shows results for the permeation studies described above.

TABLE 6

| | sampling time | Total permeation in μg/cm² |
|---|---|---|
| Sample | [h] | average of n = 3 cells |
| Glatiramer | 0.5 | 153.4 |
| acetate | 1 | 156.3 |
| solution | 1.5 | 185.9 |
| without DMSO | 2 | 265.3 |
| incubation | 3 | 268.8 |
| | 4 | 329.6 |
| Glatiramer | 0.5 | 165.8 |
| acetate | 1 | 211.5 |
| solution | 1.5 | 255.0 |
| with DMSO | 2 | 277.9 |
| incubation | 3 | 324.6 |
| | 4 | 264.7 |

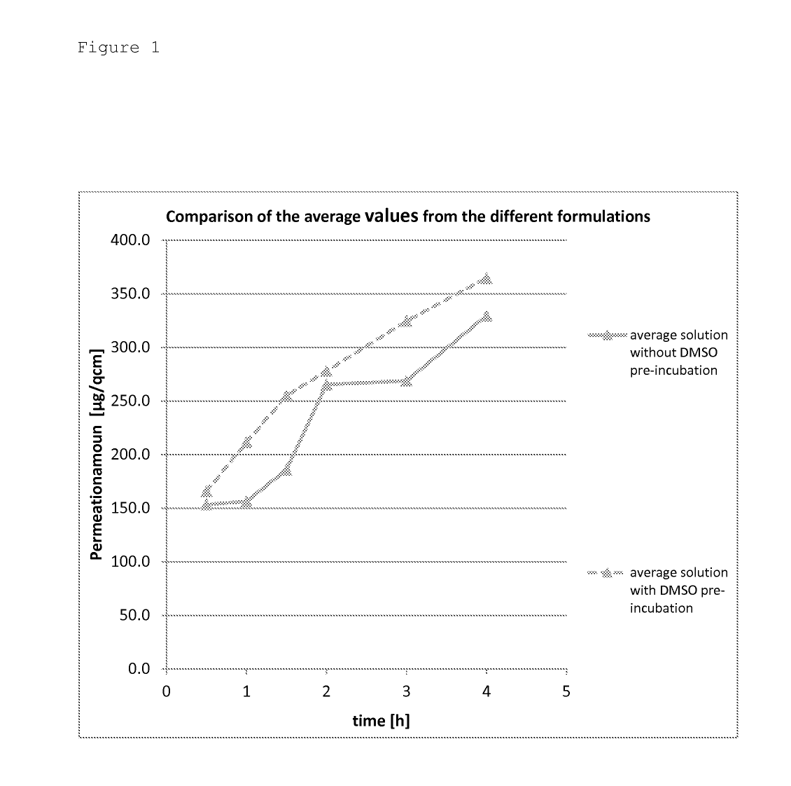

The results are shown in FIG. 1 which displays the average permeation of the different formulations. Glatiramer acetate solution without pre-incubated tissue (square markers, solid line) and glatiramer acetate solution with DMSO pre-incubated tissue (square markers, dotted line).

1. An oral patch comprising:
a) a liner; and
b) a film composition thereon, the film composition comprising glatiramer acetate in an amount from about 10 percent to about 40 percent by weight of the film composition and one or more film forming agents in a total amount from about 40 percent to about 80 percent by weight of the film composition.

2. The oral patch of claim 1, wherein the glatiramer acetate is present in the film composition in an amount from about 20 percent to about 30 percent by weight of the film composition; in an amount from about 22 percent to about 27 percent by weight of the film composition; or in an amount of about 23-25 percent by weight of the film composition.

3-4. (canceled)

5. The oral patch of claim 1, wherein the film forming agents are present in the film composition in a total amount from about 50 percent to about 70 percent by weight of the film composition.

6. The oral patch of claim 1, wherein the film composition further comprises a filler, wherein the filler is present in the film composition in an amount up to about 30 percent by weight of the film composition; in an amount from about 5 percent to about 25 percent by weight of the film composition; or in an amount from about 9 percent to about 17 percent by weight of the film composition.

7-8. (canceled)

9. The oral patch of claim 1, wherein the film composition further comprises one or more permeation enhancers, wherein the permeation enhancers are present in the film composition in a total amount up to about 10 percent by weight of the film composition; in a total amount from about 0.1 percent to about 7 percent by weight of the film composition; or in a total amount from about 0.5 percent to about 5 percent by weight of the film composition.

10-11. (canceled)

12. The oral patch of claim 1, wherein the film composition further comprises one or more flavorant, wherein the flavorants are present in the film composition in a total amount up to about 10 percent by weight of the film composition.

13. The oral patch of claim 1, wherein the film composition further comprises a pigment, wherein the pigment is present in the film composition in an amount up to about 5 percent by weight of the film composition; or in an amount of about 1 percent by weight of the film composition.

14. (canceled)

15. The oral patch of claim 1, wherein the one or more film forming agents are selected from the group consisting of Carbomer (sodium salt), polyethylene glycol, polyvinyl alcohol, microcrystalline cellulose, starch, hydroxypropyl methylcellulose and amylopectin.

16. The oral patch of claim 6, wherein the filler is selected from the group consisting of sorbitol, lactose, saccharose, sucrose, dextrose, isomalt calcium phosphate, calcium carbonate, calcium silicate, magnesium carbonate, magnesium oxide, glucopyranosyl mannitol and calcium sulfate.

17. The oral patch of claim 9, wherein the one or more permeation enhancers are selected from the group consisting of DMSO, n-Dodecyl nitrogen heterocyclic heptane-2-ketone, propylene glycol, oleic acid, isopropylmyristat and d,l-alpha-toccopherol.

18. The oral patch of claim 12, wherein the one or more flavorants are selected from the group consisting of acesulfam, saccharin-sodium, aspartame, stevia, spearmint oil, cinnamon oil, oil of wintergreen (methyl salicylate), peppermint oil, clove oil, bay oil, anise oil, eucalyptus oil, thyme oil, cedar leaf oil, oil of nutmeg, allspice, oil of sage, mace, oil of bitter almonds, cassia oil, vanilla, ethyl vanillin, citrus oils, lemon oil, orange oil, tangerine oil, lime oil, grapefruit oil, apple flavor, pear flavor, peach flavor, orange flavor, grape flavor, strawberry flavor, raspberry flavor, cherry flavor, plum flavor, pineapple flavor, apricot flavor, cinnamyl acetate, cinnamaldehyde, citral diethylacetal, dihydrocarvyl acetate, eugenyl formate and p-methylamisol.

19. The oral patch of claim 13, wherein the pigment is selected from the group consisting of titanium dioxide, talc and ferric oxide.

20. The oral patch of claim 1, wherein
a. the one or more film forming agents comprises carbomer (sodium salt), wherein the carbomer (sodium salt) is present in the film composition in an amount from about 20 percent to about 35 percent by weight of the film composition, or in an amount from about 25 percent to about 30 percent by weight of the film composition;
b. the one or more film forming agents comprises polyethylene glycol, wherein the polyethylene glycol is present in the film composition in an amount from about 0.5 percent to about 5 percent by weight of the film composition, or in an amount of about 2.5 percent by weight of the film composition;
c. the one or more film forming agents comprises polyvinyl alcohol, wherein the polyvinyl alcohol is present in the film composition in an amount from about 5 percent to about 40 percent by weight of the film composition, in an amount from about 10 percent to about 30 percent by weight of the film composition, or in an amount from about 15 percent to about 28 percent by weight of the film composition;
d. the one or more film forming agents comprises microcrystalline cellulose, wherein microcrystalline cellulose is present in the film composition in an amount from about 5 percent to about 25 percent by weight of the film composition, or in an amount of about 15 percent by weight of the film composition;
e. the one or more film forming agents comprises polyethylene glycol, wherein the polyethylene glycol is present in the film composition in an amount from about 0.5 percent to about 5 percent by weight of the film composition, or in an amount from about 2 percent to about 3 percent by weight of the film composition;
f. the one or more film forming agents comprises starch, wherein the starch is present in the film composition in an amount from about 10 percent to about 20 percent by weight of the film composition, or in an amount from about 17 percent to about 18 percent by weight of the film composition;
g. the one or more film forming agents comprises hydroxypropyl methylcellulose, wherein the hydroxypropyl methylcellulose is present in the film composition in an amount from about 10 percent to about 15 percent by weight of the film composition, or in an amount of about 13 percent by weight of the film composition; or
h. the one or more film forming agents comprises amylopectin, wherein the amylopectin is present in the film composition in an amount from about 25 percent to about 55 percent by weight of the film composition, or in an amount from about 31 percent to about 47 percent by weight of the film composition.

21-36. (canceled)

37. The oral patch of claim 9, wherein
a. the one or more permeation enhancers comprises DMSO, wherein the DMSO is present in the film composition in an amount from about from about 0.5 percent to about 5 percent by weight of the film composition; or in an amount of about 2.5 percent by weight of the film composition;
b. the one or more permeation enhancers comprises n-Dodecyl nitrogen heterocyclic heptane-2-ketone, wherein the n-Dodecyl nitrogen heterocyclic heptane-2-ketone is resent in the film composition in an amount from about from about 0.5 percent to about 5 percent by weight of the film composition, or in an amount of about 1 percent by weight of the film composition;
c. the one or more permeation enhancers comprises propylene glycol, wherein the propylene glycol is present in the film composition in an amount from about from about 0.5 percent to about 5 percent by weight of the film composition, or in an amount of about 2.5 percent by weight of the film composition; or
d. the one or more permeation enhancers comprises oleic acid, wherein the oleic acid is present in the film composition in an amount from about from about 0.5 percent to about 5 percent by weight of the film composition, or in an amount of about 1 percent by weight of the film composition.

38-44. (canceled)

45. The oral patch of claim 1, wherein
a. the one or more flavorants comprises acesulfam, wherein the acesulfam is present in the film composition in an amount from about from about 0.25 percent to about 2.5 percent by weight of the film composition, or in an amount from about 0.5 percent to about 1.5 percent by weight of the film composition;
b. the one or more flavorants comprises orange flavor, wherein the orange flavor is present in the film composition in an amount from about from about 0.25 percent to about 2.5 percent by weight of the film composition, or in an amount of about 1 percent by weight of the film composition;
c. the one or more flavorants comprises peppermint oil, wherein the peppermint oil is present in the film composition in an amount from about from about 0.25 percent to about 2.5 percent by weight of the film composition, or in an amount of about 1 percent by weight of the film composition;
d. the one or more flavorants comprises glycerol, wherein the glycerol is present in the film composition in an amount from about from about 0.5 percent to about 10 percent by weight of the film composition, or in an amount of about 4 percent by weight of the film composition; or
e. the one or more flavorants comprises spearmint oil, wherein the spearmint oil is present in the film composition in an amount from about from about 0.5 percent to about 5 percent by weight of the film composition, or in an amount of about 3 percent by weight of the film composition.

46-54. (canceled)

55. The oral patch of claim 1, wherein the patch is about 5 to about 15 cm², or is about 10 cm².

56. (canceled)

57. The oral patch of claim 1, wherein the patch contains about 20 mg to about 100 mg glatiramer acetate, or contains about 25 mg glatiramer acetate.

58. (canceled)

59. The oral patch of claim 1, wherein the liner is a polyethylene terephthalate (PET) liner.

60. An oral patch comprising:
a) a PET liner; and
b) a film composition thereon, the film composition comprising glatiramer acetate in an amount of about 25 percent by weight of the film composition and film forming agents, wherein the film forming agents comprise:
i. carbomer (sodium salt) present in the film composition in an amount of about 25.5 percent by weight of the film composition;
ii. polyethylene glycol present in the film composition in an amount of about 2.5 percent by weight of the film composition;
iii. polyvinyl alcohol present in the film composition in an amount of about 10 percent by weight of the film composition; and
iv. microcrystalline cellulose present in the film composition in an amount of about 15 percent by weight of the film composition,
wherein the film composition further comprises a filler which comprises sorbitol present in the film composition in an amount of about 17 percent by weight of the film composition,
wherein the film composition further comprises a permeation enhancer which comprises DMSO present in the film composition in an amount of about 2.5 percent by weight of the film composition,
wherein the film composition further comprises flavorants which comprise:
a) acesulfam present in the film composition in an amount of about 1.5 percent by weight of the film composition; and
b) orange flavor present in the film composition in an amount of about 1.5 percent by weight of the film composition,
wherein the patch is about 10 cm²,
wherein the patch contains about 25 mg glatiramer acetate.

61-62. (canceled)

63. An oral patch comprising:
a) a PET liner; and
b) a film composition thereon, the film composition comprising glatiramer acetate in an amount of about 24 percent by weight of the film composition and film forming agents, wherein the film forming agents comprise:
i. polyvinyl alcohol present in the film composition in an amount of about 10 percent by weight of the film composition;
ii. polyethylene glycol present in the film composition in an amount of about 2.5 percent by weight of the film composition; and
iii. amylopectin present in the film composition in an amount of about 46 percent by weight of the film composition,
wherein the film composition further comprises a filler which comprises sorbitol present in the film composition in an amount of about 10 percent by weight of the film composition,
wherein the film composition further comprises permeation enhancers which comprise:
a) propylene glycol present in the film composition in an amount of about 2.5 percent by weight of the film composition; and
b) oleic acid present in the film composition in an amount of about 1 percent by weight of the film composition,
wherein the film composition further comprises flavorants which comprise:
a) acesulfam present in the film composition in an amount of about 1.5 percent by weight of the film composition; and
b) spearmint flavor present in the film composition in an amount of about 3 percent by weight of the film composition,
wherein the patch is about 10 cm²,
wherein the patch contains about 25 mg glatiramer acetate.

64. (canceled)
